(19) United States
(12) Patent Application Publication
(10) Pub. No.: US 2007/0249277 A1
(43) Pub. Date: Oct. 25, 2007

(54) CONSTANT-TEMPERATURE BOX CAPABLE OF CONTROLLING TEMPERATURE AND AIRFLOW

(76) Inventor: Guei-Han Chou, Taipei City (TW)

Correspondence Address: NORTH AMERICA INTELLECTUAL PROPERTY CORPORATION, P.O. BOX 506, MERRIFIELD, VA, 22116, US

(21) Appl. No.: 11/460,252

(22) Filed: Jul. 27, 2006

(30) Foreign Application Priority Data
Apr. 21, 2006 (TW) 095206836

(51) Int. Cl. B08B 15/00; C12M 1/00; A01G 31/00

(52) U.S. Cl. 454/49; 435/303.1; 47/59 R

(57) ABSTRACT

Division plates inside a constant-temperature box form an inner box for positioning an electronic device to be tested. Airflow guiding devices, temperature regulators, and airflow regulators are positioned inside the constant-temperature box. The airflow guiding devices direct air from air vents near the bottom of the constant-temperature box into the inner box. The temperature regulators adjust air temperature between the inner box and the constant-temperature box. The airflow regulators regulate air directed by the airflow guiding devices so as to stabilize the speed of airflow inside the inner box. Therefore, when the electronic device is tested, the test result will not be influenced by airflow.

### BACKGROUND OF THE INVENTION

1. Field of the Invention

The present invention relates to a constant-temperature box, and more particularly, to a constant-temperature box capable of controlling temperature and airflow.

2. Description of the Prior Art

The development of electronic products requires numerous testing phases, such as experiments relating to the operating performance of electronic products under different temperatures. In most cases, influential factors such as speed of airflow must be properly controlled for obtaining a reasonable experimental result.

Please refer to FIG. 1. FIG. 1 is a perspective diagram showing a prior art constant-temperature box 1. As shown in FIG. 1, the constant-temperature box 1 includes a plurality of lamps 10 and a plurality of fans 12 therein. The bottom of the constant-temperature box 1 includes an air vent 14 for providing a ventilation path to the external environment. The lamps 10 are utilized to increase the temperature inside the constant-temperature box 1, thereby simulating an environment with different temperatures. Since the position of the lamps 10 is fixed, the heat generated by the lamps 10 is not able to evenly spread to the entire constant-temperature box 1. Hence, the fan 12 is rotated to facilitate air circulation and spread the heat generated by the lamps 10. Nevertheless, the air generated by the rotation of the fan 12 ultimately becomes a significant factor for influencing the result of the experiment.

### SUMMARY OF THE INVENTION

According to the present invention, a constant-temperature box includes: a housing, a separating plate, a first airflow guiding device, a first temperature regulator, a temperature sensor, a second airflow guiding device, a first airflow regulator, a second airflow regulator, and a controller. The housing has a first opening located on a first side of the housing, in which the first opening is close to the bottom of the first side. The separating plate is disposed in the housing and close to the first side, in which the first separating plate further comprises a second opening correspond to the first opening. The first airflow guiding device is disposed between the first side of the housing and the first separating plate for directing the air between the first side of the housing and the first separating plate upward. The first temperature regulator is disposed between the first side of the housing and the first separating plate for regulating the air temperature between the first side and the first separating plate. The temperature sensor is disposed in the housing for sensing the temperature within the housing. The second airflow guiding device is disposed in the housing and close to the upper side of the housing for directing the air close to the upper side of the housing downward. The first airflow regulator is disposed below the second airflow guiding device for regulating the air directed by the second airflow guiding device. The second airflow regulator is disposed below the first airflow regulator for supporting an object. The controller is electrically connected to the first airflow guiding device, the second airflow guiding device, the first temperature regulator, and the temperature sensor, for controlling the operation of the first airflow guiding device, the second airflow guiding device, and the first temperature regulator according to the temperature detected by the temperature sensor.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a perspective diagram showing a prior art constant-temperature box.

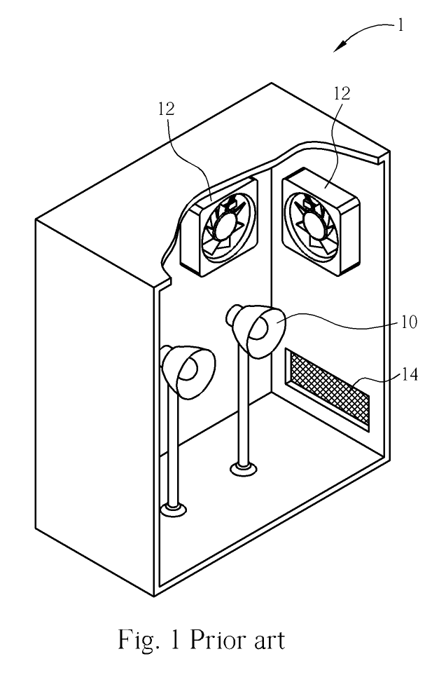

FIG. 2 is a perspective diagram showing a constant-temperature box according to an embodiment of the present invention.

### DETAILED DESCRIPTION

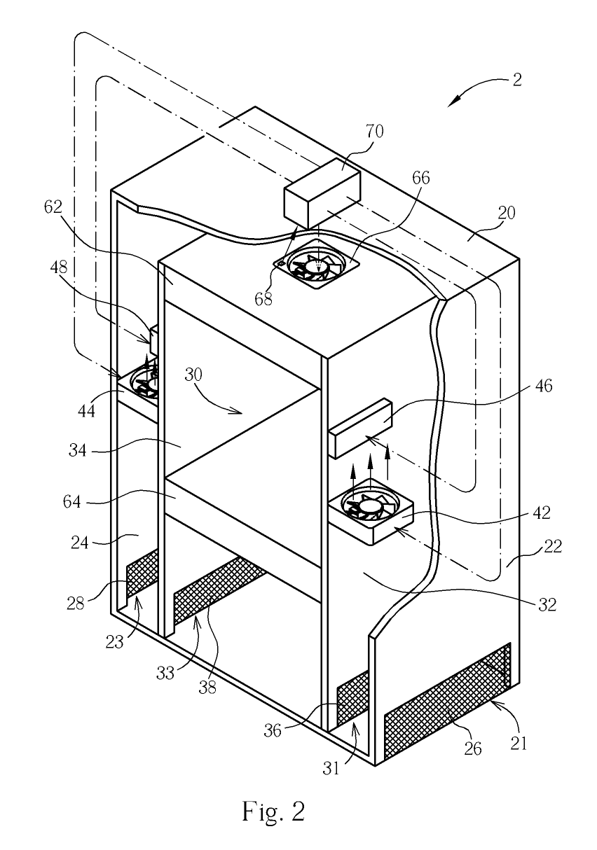

Please refer to FIG. 2. FIG. 2 is a perspective diagram showing a constant-temperature box 2 according to an embodiment of the present invention. As shown in FIG. 2, the constant-temperature box 2 includes a housing 20 and an inner box 30, in which the inner box 30 is formed by two separating plates 32 and 34. The housing 30 includes a first side 22 and a second side 24, and the bottom of the first side 22 and the second side 24 includes an opening 21 and an opening 23 respectively. The bottom of the separating plates 32 and 34 corresponding to the openings 21 and 23 also includes an opening 31 and an opening 33. A plurality of meshed airflow regulating plates 26, 28, 36, and 38 is disposed in each of the openings 21, 23, 31, and 33.

An airflow guiding device 42 and a temperature regulator 46 are disposed between the first side 22 of the housing 20 and the separating plate 32 of the inner box 30. Similarly, an airflow guiding device 44 and a temperature regulator 48 are disposed between the second side 24 of the housing 20 and the separating plate 34 of the inner box 30. An airflow regulator 62, an airflow guiding device 66, and a temperature sensor 68 are disposed on the inner box 30, and an airflow regulator 64 is positioned in the space above the openings 36 and 38 for supporting an object, such as an electronic product to be tested. Additionally, a controller 70 is electrically connected to the airflow guiding device 42, 44, and 66, the temperature regulators 46 and 48, and the temperature sensor 68.

The following description offers additional details about the operation of the constant-temperature box 2 of the present invention. First, the meshed airflow regulating plates 26 and 28 disposed in the openings 21 and 23 are utilized to regulate the air as it enters from the openings 21 and 23. Alternatively, the present invention also allows the air to directly pass through the openings 21 and 23 and this is accomplished by not installing any of the meshed airflow regulating plates 26 and 28 in any of the opening 21 and 23. In this case, the air can be regulated before entering the inner box 30. The mechanism pertaining to this design is explained in a later section of the present disclosure.

Next, the airflow guiding devices 42 and 44 will guide the bottom region air upward, and the temperature regulator 46 and 48 will adjust the temperature between the inner box 30 and the constant-temperature box 2. Suppose the temperature required by the experiment is 35° C., which is higher than the temperature of the external environment, the temperature regulators 46 and 48 will heat the air to 35° C. After the air heated by the temperature regulators 46 and 48 reaches the top region of the inner box 30, the temperature sensor 68 will detect the circulating air, and the controller 70 will control the rotational speed of the airflow guiding devices 42, 44, and 66 according to the temperature detected by the temperature sensor 68 and also control the temperature regulation manipulated by the temperature regulator 46 and 48.

Preferably, the airflow guiding device 66 will guide the warm air circulating in the upper region of the inner box 30 downward and through the airflow regulator 62 to decrease the speed caused by the airflow guiding device 66 and reduce the air-circulation effect influencing the experiment. After passing through the electronic product being tested, the warm air is expelled from the bottom of the inner box 30 through the airflow regulator 64. Next, the warm air expelled from the bottom of the inner box 30 is mixed with the cold air entering the constant-temperature box 2, and directed through the temperature regulators 46 and 48 via the airflow guiding devices 42 and 44.

Additionally, the temperature regulators 46 and 48 include heaters and coolers for selectively heating or cooling the air temperature, in which the temperature regulators 46 and 48 can be positioned above or below the airflow guiding devices 42 and 44. In other words, the present invention is able to utilize the airflow guiding devices 42 and 44 to direct the air upward and then utilize the temperature regulators 46 and 48 to adjust the air temperature, or utilize the temperature regulators 46 and 48 to adjust the air temperature while directing the air upward through the airflow guiding devices 42 and 44. It should be noted that the airflow guiding devices 42 can be implemented by fans or other devices capable of directing airflow, and the airflow regulators 62 and 64 can be implemented by meshed airflow regulating plates composed of any materials or any other devices capable of regulating airflow.

Preferably, the constant-temperature box of the present invention utilizes a design of having an external housing and an inner box, in which a plurality of airflow guiding devices and temperature regulators is disposed between the inner box and the housing to adjust the temperature within the box and create a fixed-temperature condition required by the experiment. Before entering the inner box, the speed of the air is reduced by the airflow regulators to significantly reduce the influence to the experiment caused by air circulation. Additionally, the controller is able to adaptively adjust the temperature and the speed of air circulation within the constant-temperature box according to the temperature detected by the temperature sensor, thereby increasing the performance of the constant-temperature box.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A constant-temperature box, comprising:
a housing, having a first opening located on a first side of the housing, wherein the first opening is close to the bottom of the first side;
a first separating plate, disposed in the housing and close to the first side of the housing, wherein the first separating plate comprises a second opening disposed at a location corresponding to the first opening;
a first airflow guiding device, disposed between the first side of the housing and the first separating plate, for directing the air between the first side of the housing and the first separating plate upward;
a first temperature regulator, disposed between the first side of the housing and the first separating plate, for regulating the air temperature between the first side of the housing and the first separating plate;
a temperature sensor, disposed in the housing, for sensing the temperature within the housing;
a second airflow guiding device, disposed in the housing and close to the upper side of the housing, for directing the air close to the upper side of the housing downward;
a first airflow regulator, disposed below the second airflow guiding device, for regulating the air directed by the second airflow guiding device;
a second airflow regulator, disposed below the first airflow regulator, for supporting an object; and
a controller, electrically connected to the first airflow guiding device, the second airflow guiding device, the first temperature regulator, and the temperature sensor, for controlling the operation of the first airflow guiding device, the second airflow guiding device, and the first temperature regulator according to the temperature detected by the temperature sensor.

2. The constant-temperature box of claim 1, wherein a second side of the housing is parallel to the first side of the housing; the second side comprises a third opening located close to the bottom of the second side; and the constant-temperature box further comprises a second separating plate disposed in the housing and close to the second side, wherein the second separating plate comprises a fourth opening disposed at a location corresponding to the third opening.

3. The constant-temperature box of claim 2 further comprising:
a plurality of meshed airflow regulating plates disposed in the first opening, the second opening, the third opening, and the fourth opening, respectively.

4. The constant-temperature box of claim 2 further comprising:
a third airflow guiding device, disposed between the second side of the housing and the second separating plate, for directing the air between the second side of the housing and the second separating plate upward; and
a second temperature regulator, disposed between the second side and the second separating plate, for adjusting the air temperature between the second side of the housing and the second separating plate.

5. The constant-temperature box of claim 4, wherein the second temperature regulator comprises a heater for heating the air between the second side of the housing and the second separating plate.

6. The constant-temperature box of claim 4, wherein the second temperature regulator comprises a cooler for cooling the air between the second side of the housing and the second separating plate.

7. The constant-temperature box of claim 4, wherein the second temperature regulator is disposed above the third airflow guiding device.

8. The constant-temperature box of claim 4, wherein the second temperature regulator is disposed below the third airflow guiding device.

9. The constant-temperature box of claim 4, wherein the third airflow guiding device is a fan.

10. The constant-temperature box of claim 1, wherein each of the first airflow regulator and the second airflow regulator comprise a meshed airflow regulating plate.

11. The constant-temperature box of claim 1, wherein the first temperature regulator comprises a heater for heating the air between the first side of the housing and the first separating plate.

12. The constant-temperature box of claim 1, wherein the first temperature regulator comprises a cooler for cooling the air between the first side of the housing and the first separating plate.

13. The constant-temperature box of claim 1, wherein each of the first airflow guiding device and the second airflow guiding device comprises a fan.

14. The constant-temperature box of claim 1, wherein the first temperature regulator is disposed above the first airflow guiding device.

15. The constant-temperature box of claim 1, wherein the first temperature regulator is disposed below the first airflow guiding device.
